(19) United States
(12) Patent Application Publication
(10) Pub. No.: US 2023/0285497 A1
(43) Pub. Date: Sep. 14, 2023

(54) ATOPIC DERMATITIS THERAPY WITH KAPPA OPIOID RECEPTOR AGONIST AS ADJUNCT TO TOPICAL CORTICOSTEROID

(71) Applicant: Cara Therapeutics, Inc., Stamford, CT (US)

(72) Inventors: Joana Goncalves, Greenwich, CT (US); Kristine E. Nograles, Green Brook, NJ (US); Derek T. Chalmers, Riverside, CT (US)

(73) Assignee: Cara therapeutics, Inc., Stamford, CT (US)

(21) Appl. No.: 17/731,312

(22) Filed: Apr. 28, 2022

Related U.S. Application Data
(60) Provisional application No. 63/319,025, filed on Mar. 11, 2022.

(51) Int. Cl. A61K 38/07; A61K 31/573; A61K 45/06

(52) U.S. Cl. CPC A61K 38/07; A61K 31/573; A61K 45/06

(57) ABSTRACT

The invention relates to a method of treatment of atopic dermatitis in a patient, the method includes providing a course of treatment comprising orally administering an effective amount of a kappa opioid receptor agonist (KORA) to the patient and applying an effective amount of a topical corticosteroid (TCS) to an area of the patient affected by the atopic dermatitis.

Pruritus, or itch, is defined as an unpleasant sensation that provokes the desire to scratch, which can range from a mild annoyance to an intractable, disabling condition. Chronic pruritus is a significant unmet need with no robustly efficacious or pruritus-targeting treatment options. It is estimated that about one in every eight people globally suffer from chronic pruritus. The condition is often under-reported by patients and often under-treated. Pruritus has a significant negative impact on patients' quality of life.

Underlying diseases leading to chronic pruritus are primarily systemic diseases, dermatological diseases, and neurological diseases. Each disease category includes numerous individual diseases with which chronic pruritus is associated or a key symptomatic feature of that particular condition (See Stander S. et al., Clinical Classification of Itch: a Position Paper of the International Forum for the Study of Itch; Acta Derm Venereol 2007; 87: 291-294).

Certain systemic diseases known to cause pruritus include endocrine and metabolic diseases such as chronic kidney disease and chronic liver disease like primary biliary cholangitis, infectious disease, hematologic and lymphoproliferative diseases, visceral neoplasms and drug-induced pruritus.

Dermatological diseases known to cause pruritus include inflammatory dermatoses—such as atopic dermatitis, infectious dermatoses, autoimmune dermatoses, neoplasms, genodermatoses, and dermatoses of pregnancy. Neurological diseases known to have pruritus include notalgia paresthetica, brachioradial pruritus and post-herpetic neuralgia.

Effective treatment of pruritus remains a substantial unmet need. In the U.S. approximately 23 million patients diagnosed with diseases known to trigger chronic pruritus receive prescriptions for an anti-pruritic agent such as a corticosteroid, an antihistamine, any of a number of select antidepressants, counterirritants, bile acid sequestrants, rifampin, narcotic antagonists, partial agonists, and topical immune-modulators. However, none of these treatments provide complete remission of the pruritus.

### SUMMARY OF THE INVENTION

The invention provides a method of treating or inhibiting atopic dermatitis (AD) in a patient in need thereof, the method comprising administering a kappa opioid receptor agonist (KORA) and a topical corticosteroid (TCS) to the patient. Surprisingly, the inventors find that the administration of a combination of a KORA and a TCS to a patient suffering from atopic dermatitis provides a synergistic treatment benefit in reducing pruritus.

In one embodiment, the invention provides a method of treating or inhibiting atopic dermatitis in patient in need thereof, the method comprising administering a kappa opioid receptor agonist (KORA) and a topical corticosteroid (TCS) to the patient, wherein the KORA is nalfurafine or asimadoline.

In another embodiment, the invention provides a method of treating or inhibiting atopic dermatitis in patient in need thereof, the method comprising administering a KORA and a TCS to the patient, wherein the kappa opioid receptor agonist is peripherally restricted.

In another embodiment the invention provides a method of treating or inhibiting atopic dermatitis in patient in need thereof, the method comprising administering a KORA and a TCS to the patient, wherein the kappa opioid receptor agonist comprises D-amino acids.

In still another embodiment, the invention provides a method of treating or inhibiting atopic dermatitis in patient in need thereof, the method comprising administering a KORA and a TCS to the patient, wherein the KORA comprising D-amino acids is selected from the group consisting of JT07, Compound 8 of U.S. Pat. No. 11,014,964, and Compound 17 of U.S. Pat. No. 11,014,964.

In another embodiment the invention provides a method of treating or inhibiting atopic dermatitis in patient in need thereof, the method comprising administering a KORA and a TCS to the patient, wherein the KORA comprising D-amino acids is Difelikefalin.

In another embodiment the invention provides a method of treating or inhibiting atopic dermatitis in patient in need thereof, the method comprising administering a KORA and a TCS to the patient, wherein the KORA is administered intravenously.

In another embodiment the invention provides a method of treating or inhibiting atopic dermatitis in patient in need thereof, the method comprising administering a KORA and a TCS to the patient, wherein the kappa opioid receptor agonist is administered orally or topically.

In another embodiment the invention provides a method of treating or inhibiting atopic dermatitis or inflammatory dermatitis in a patient in need thereof, the method comprising administering a KORA and a TCS to the patient. In one embodiment, the inflammatory dermatitis comprises contact dermatitis, psoriasis or seborrhetic dermatitis.

In yet another embodiment, the invention provides a method of treating or inhibiting pruritus associated with atopic dermatitis in a patient, the method comprising administering a KORA and a TCS to the patient.

In still another embodiment, the invention provides a method of treating or inhibiting skin lesions associated with atopic dermatitis in a patient, the method comprising administering a KORA and a TCS to the patient.

The topical corticosteroid (TCS) useful in the course of treatment of atopic dermatitis in patient in need thereof, can be any suitable corticosteroid, such as, for example, and without limitation, a high potency corticosteroid, a medium potency corticosteroid, a lower-medium potency corticosteroid, or a low potency medium potency corticosteroid.

For example, a very high potency medium potency corticosteroid can be any corticosteroid selected from the group consisting of augmented betamethasone dipropionate, diflurasone diacetate and halobetasol propionate.

In another example, a high potency medium potency corticosteroid can be any corticosteroid selected from the group consisting of amcinonide, augmented betamethasone dipropionate, desoximetasone, diflurasone diacetate, flurocinonide, halcononide, mometasone furoate and trioamcinolone acetonide.

In still another example, a medium potency medium potency corticosteroid can be any corticosteroid selected from the group consisting of betamethasone valerate, clocortolone pivalate, desoximetasone, flucinolone acetonide, flurandrenolide, fluticasone propionate, mometasone furoate and triamcinolone acetonide.

In another example, a lower-medium potency corticosteroid can be any corticosteroid selected from the group consisting of hydrocortisone butyrate, hydrocortisone valerate and prednicarbate.

In another example, a low potency corticosteroid can be any corticosteroid selected from the group consisting of alclometasone dipropionate, desonide and fluocinolone acetonide.

In another example, a lowest potency corticosteroid can be any corticosteroid selected from the group consisting of dexamethasone, hydrocortisone and hydrocortisoneacetate.

In another embodiment, the invention provides a method of treating or inhibiting atopic dermatitis in patient in need thereof, the method comprising administering a KORA and a TCS to the patient, wherein the TCS is selected from the group consisting of prednisone, prednisolone, methylprednisolone, cortisone, hydrocortisone, dexamethasone, triamcinolone, beclomethasone and clobetasone.

The invention further provides a method of treatment of a patient suffering from atopic dermatitis, the method includes executing a course of treatment comprising: orally administering an effective amount of a kappa opioid receptor agonist (KORA) to the patient and applying an effective amount of a topical corticosteroid (TCS) to an area of the patient affected by the atopic dermatitis. The TCS can be in the form of an ointment, a cream, a gel, a lotion, a foam or a solution.

The method of treatment of the invention includes executing a course of treatment to a patient suffering from atopic dermatitis, the method comprising: orally administering an effective amount of a kappa opioid receptor agonist (KORA) to the patient and applying an effective amount of a topical corticosteroid (TCS) to an area of the patient affected by the atopic dermatitis. The KORA can be any suitable kappa opioid receptor agonist, such as for instance, and without limitation a KORA selected from the group consisting of Difelikefalin, asimadoline, nalfurafine, JT07, Compound 8 of U.S. Pat. No. 11,014,964, and Compound 17 of U.S. Pat. No. 11,014,964.

In one embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the oral administration of the KORA to the patient and the application of the TCS to an area of the patient affected by the atopic dermatitis occurs on the same day.

In another embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the oral administration of the KORA to the patient occurs one or more times per day and the application of the TCS to an area of the patient affected by the atopic dermatitis occurs one or more times per day.

In another embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the oral administration of the KORA to the patient and the application of the TCS to an area of the patient affected by the atopic dermatitis each occur twice per day.

In another embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the oral administration of the KORA to the patient and the application of the TCS to an area of the patient affected by the atopic dermatitis each occur one or more times per day for from 1-7 days.

In another embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the oral administration of the KORA to the patient and the application of the TCS to an area of the patient affected by the atopic dermatitis each occur one or more times per day for from 8-84 days.

In another embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the oral administration of the KORA to the patient and the application of the TCS to an area of the patient affected by the atopic dermatitis each occur one or more times per day for 84 days or more.

In still another embodiment, the method includes executing a course of treatment to a patient suffering from atopic dermatitis, the method comprising: orally administering an effective amount of a KORA to the patient and applying an effective amount of TCS to an area of the patient affected by the atopic dermatitis, wherein the oral administration of the KORA to the patient and the application of the TCS to an area of the patient affected by the atopic dermatitis each occur one or more times per day for one or more days until control of the atopic dermatitis of the affected area is achieved and subsequently as needed to treat recurrences of the disease. In another embodiment, the above course of treatment is for from 1-7 days. In still another embodiment, the above course of treatment is for from 8-84 days. In yet another embodiment, the above course of treatment is for 84 or more days.

In another embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the KORA is administered in an oral formulation selected from an oral formulation selected from a powder, a tablet, a capsule, a gelcap, an amorphous solid, a crystalline form, a liquid, a slurry and a gel.

In another embodiment, the method includes executing a course of treatment to a patient suffering from atopic dermatitis, the method comprising: orally administering an effective amount of a KORA to the patient and applying an effective amount of a TCS to an area of the patient affected by the atopic dermatitis, wherein the oral formulation comprises from 0.01-10.0 mg Difelikefalin. In another embodiment, the oral formulation comprises from 0.1-5.0 mg Difelikefalin. In still another embodiment, the oral formulation comprises from 0.2-1.0 mg Difelikefalin. In yet another embodiment, the oral formulation comprises from 0.25-0.5 mg Difelikefalin.

In another embodiment, the method includes executing a course of treatment to a patient suffering from atopic dermatitis, the method comprising: orally administering an effective amount of a KORA, such as for instance, Difelikefalin to the patient and applying an effective amount of a TCS to an area of the patient affected by the atopic dermatitis, wherein the TCS is selected from the group consisting of betamethasone valerate, clocortolone pivalate, desoximetasone, fluocinolone acetonide, flurandrenolide, fluticasone propionate, mometasone furoate and triamcinolone acetonide.

### BRIEF DESCRIPTION OF THE FIGURES

FIG. 1 shows the percent achieving at least a 4 point improvement of itch-numerical rating score (I-NRS) in subjects having atopic dermatitis after twelve (12) weeks twice daily treatment with placebo (N=79 subjects), 0.25 mg Difelikefalin (N=50), 0.50 mg Difelikefalin (N=82), 0.25 mg Difelikefalin (N=46). All subjects (N=178) had a baseline validated Investigator's Global Assessment scale for AD of greater than or equal to 2 and a body surface area involvement of less than or equal to 30 percent and moderate-to-severe AD-related pruritus with a mean baseline Itch Numerical Rating Scale score of greater than or equal to 5.0 (I-NRS≥5.0). The greater than or equal to 4 point improvement I-NRS of subjects receiving 0.25 mg Difelikefalin was 34%; 0.50 mg Difelikefalin was 33% (p=0.046), 1.0 mg Difelikefalin was 28%, All Doses was 32% (P=0.033), and for subjects receiving Placebo the improvement was 19%.

### DETAILED DESCRIPTION OF THE INVENTION

The invention provides a method of treating or inhibiting atopic dermatitis in patient in need thereof, the method comprising administering a kappa opioid receptor agonist and a topical corticosteroid to the patient. One common symptom of atopic dermatitis is pruritus.

Pruritus, commonly referred to as itch, is a symptom in many forms of dermatitis. Other heterogenous symptoms include dryness, redness, oozing/weeping, skin pain, sleep disturbance, anxiety and depression. Pruritus originates in the epidermis and dermal-epidermal junction and is transmitted by itch-selective sensory neuron C fibers, or pruriceptors. Some of these fibers are sensitive to histamine whereas others are not. There is evidence for histamine-insensitive C fibers that are activated by numerous itch-inducing substances or pruritogens, many of which initiate signals through interaction with specific G-protein-coupled receptors. In addition, there is increasing evidence for the differential involvement of these systems in various forms of itch which may involve disease-specific pruritogens. For example, chronic pruritus associated with kidney failure is thought to involve complex interactions among peripheral cells (T cells, mast cells, neutrophils, eosinophils, and keratinocytes) and histamine-insensitive nerve fibers, involving increased release of cytokines, proteases, and neuropeptides, interacting with multiple receptors that lead to exacerbation of itch. These peripheral cell types express kappa opioid receptors, or KORs, which can regulate the release of these pruritogenic substances, while the KORs on C fibers are thought to regulate their response to these pruritogens. Because KORs are expressed in peripheral tissues including skin cells, there is a potential to modulate itch signals peripherally without impacting the central KORs. The itch-sensitive sensory nerve fibers transmit signals to the cell bodies in the dorsal root ganglia, which send fibers to enter the spinal cord. Itch signals then ascend via the spinothalamic tract to multiple brain areas for sensory processing and interactions with cognitive and other systems. Additionally, the activation of kappa receptors via an agonist is thought to reduce itching by functionally counteracting increased mu opioid receptor activity which is suggested to be associated with several chronic forms of pruritus. Activation of the mu opioid receptor in the brain and in the peripheral nerve endings results in itching, while non-selective mu opioid antagonists can inhibit itching. KOR stimulation inhibits the effects of mu receptor activation both centrally and peripherally. KOR stimulation is mediated by kappa opioid receptor agonists (KORAs).

Opioids are the most widely used and effective analgesic medications for the treatment of pain and related disorders. Opiates have been used for thousands of years for the treatment of pain, and in the last century huge strides have been made in the development of opioids derived from naturally occurring opiates in pharmacology and medicinal chemistry. Opioids are also often used in the treatment of numerous other disorders, including diarrhea, cough, postoperative pain, and cancer. The four major classes of opioid receptors are the μ receptor (mu: MOP), the δ receptor (delta: DOP), the κ receptor (kappa: KOP) and the nociception receptor (NOR). These receptors are G-protein coupled and activate inhibitory G proteins formed as homo- and heterodimeric complexes that signal to kinase cascades and are in a scaffold with a variety of proteins. The addictive pharmacological actions of morphine attributed are mediated uniquely through MORs, while DORs have been shown to mediate anxiolytic and anti-depressive properties. KORs are normally activated in vivo by dynorphins produced in parts of the nervous system, including the hypothalamus, the striatum, the hippocampus and the spinal chord in functions related to learning, memory, emotional control, stress and pain. KOR signaling associated with analgesia is also less commonly related to allodynia or hyperalgesia (heightened sensitivity or extreme response to pain due to mild/non-noxious stimulation).

### Kappa Opioid Receptor Agonists (KORAs).

Kappa opioid receptors (KORs) are found on certain neurons and on circulating immune cells. Primary afferent neurons are known to be inhibited by activation of kappa opioid receptor (KOR) signaling consistent with the finding that KORs are expressed in several populations of afferent neurons, also a subset of peptidergic sensory neurons and low threshold mechanoreceptors with endings around hair follicles. KOR signaling acts in the central nervous system to inhibit calcium ion (Ca²⁺) flux and in the periphery, KOR signaling inhibits neurogenic inflammation and nociceptor sensitization by inflammatory mediators. Furthermore, peripherally restricted KOR agonists (KORAs) selectively reduce pain and itch as well as mechanical hypersensitivity associated with surgical incision. (Snyder et al Kappa Opioid Receptor Distribution and Function in Primary afferents (2018) Neuron 99: 1274-1288).

Difelikefalin is a synthetic peptide with a single stereoisomer and is present as an acetate salt in KORSUVA™ available in the U.S. Difelikefalin acetate is a white to off-white powder with a molecular weight of 679.4 g/mol (monoisotopic; free base). It is soluble in water. The chemical name of Difelikefalin acetate is 4-amino-1-(D-phenylalanyl-D-phenylalanyl-D-leucyl-D-lysyl)piperidine-4-carboxylic acid, acetate salt.

Difelikefalin, is a highly selective, predominantly peripherally acting KOR agonist, or KORA with no identified off-target activity. The physiochemical properties of Difelikefalin (e.g., hydrophilic, synthetic D-amino acid peptide with high polar surface area and charge at physiological pH) minimize passive diffusion or active transport through the blood-brain barrier, thus limiting penetration into the brain. Data indicate that Difelikefalin preferentially activates KORs expressed outside of the central nervous system (CNS), which should mitigate side effects, especially dysphoria and psychomimetic effects, that are associated with the activation of centrally located KORs. As a KORA, Difelikefalin's mechanism of action is mediated through down regulation of the itch sensation by acting on the peripheral neurons responsible for sensing pruritus. In other words, Difelikefalin disrupts the itch sensation of chronic pruritus at the nerve level. In addition, Difelikefalin acts on KORs expressed on a range of activated immune cells to subsequently block the release of pruritogenic cytokines. Without being bound by theory it is believed that by acting on peripheral nerves that sense pruritus, Difelikefalin acts to reduce pruritus regardless of the underlying disease state.

### Mechanism of Atopic Dermatitis

Atopic dermatitis is a chronic pruritic inflammatory disease often occurring in children. The conventional paradigm of atopic dermatitis pathogenesis and associated pruritus holds that immune cells are activated to produce pro-inflammatory cytokines through cytokine receptors that in turn transduce a signal through the JAK/STAT pathway leading to inflammation of the skin characteristic of atopic dermatitis. Pro-inflammatory cytokines are over-expressed in atopic dermatitis Mu opioid pathways activate this pro-inflammatory cytokine signal transduction, whereas kappa opioids inhibit or down regulate this signal reducing the dermatitis and associated pruritis.

Pruritus is considered to be a hallmark of AD, such that AD is often described as “the itch that rashes” (Boguniewicz M. Atopic dermatitis: beyond the itch that rashes. Immunol Allergy Clin North Am 2005; 25(2):333-351, vii). It is also considered the most debilitating symptom of AD that drives the “itch-scratch cycle” further aggravating damage to the skin barrier and results in sleeplessness, fatigue, and poor quality of life (Mack M R and Kim B S. The Itch-Scratch Cycle: A Neuroimmune Perspective. Trends Immunol 2018; 39(12):980-991; Darsow U. et al. Pruritus and atopic dermatitis. Clin Rev Allergy Immunol 2011; 41(3):237-244). Chronic pruritus in AD is mediated by a complex interplay between keratinocytes, cutaneous nerve fibers, pruritogenic molecules, and the peripheral and central nervous system (Pavlis J and Yosipovitch G. Management of Itch in Atopic Dermatitis. Am J Clin Dermatol 2018; 19(3):319-332). An imbalance in the epidermal opioid system has also been postulated to play a role in the modulation of pruritus in AD (Tominaga M, et al. Possible roles of epidermal opioid systems in pruritus of atopic dermatitis. J Invest Dermatol 2007; 127(9):2228-2235).

Current guidelines for treatment of atopic dermatitis by allergists and dermatologists share similar recommended interventions as well as additional distinct approaches (Eichenfield, L. F. et al. J. Allergy Clin. Immunol. April 2017 S49-S57).

These treatments have mixed results and include bathing with warm water and applying moisturizers to increase skin hydration; application of topical corticosteroids, vitamin D therapy, administration of antimicrobials, application of calcineurin inhibitors such as tacrolimus, in an ointment formulation and various additional topical therapies including antihistamines as well as systemic immunosuppressants; the latter being strongly discouraged for long term use or for use in children (Eichenfield, L. F. et al J. Amer. Acad. Dermatol. July 2014 vol: 71(1): 116-132.).

Corticosteroids are produced naturally in the adrenal gland and normally regulate stress and immune system responses. Treatment with topical corticosteroids is a first line therapy recommended by allergists when non-pharmacologic interventions have failed while cautioning against use of topical corticosteroids in thin-skinned areas, whereas allergists tend to emphasize avoidance of undertreatment and prescribe topical corticosteroids more frequently.

Topical Corticosteroids (TCSs) are generally classified in seven potency classes (I-VII) percentage concentrations are percent weight for weight (w/w) of the TCS carrier medium:
I. Very High Potency: For example,
   Augmented betamethasone dipropionate (Ointment 0.05%); Clobetasol propionate (Cream, foam, ointment 0.05%); Diflorasone diacetate (Ointment 0.05%); Halobetasol propionate (Cream, ointment 0.05%);
II. High Potency: For example,
   Amcinonide (Cream, lotion, ointment 0.1%); Augmented betamethasone dipropionate (Cream 0.05%); Betamethasone dipropionate (Cream, foam, ointment, solution 0.05%); Desoximetasone (Cream, ointment 0.25%); Desoximetasone (Gel 0.05%); Diflurasone diacetate (Cream 0.05%); Flurocinonide (Cream, gel, ointment, solution 0.05%); Halcinonide (Cream, ointment 0.1%); Mometasone furoate (Ointment 0.1%); Triamcinolone acetonide (Cream, ointment 0.5%);
III-IV. Medium Potency: For example,
   Betamethasone valerate (Cream, foam, lotion, ointment 0.05%); Clocortolone pivalate (Cream 0.1%); Desoximetasone (Cream, ointment 0.1%); Fluocinolone acetonide (Cream, ointment 0.025%); Flurandrenolide (Cream, ointment 0.5%); Fluticasone propionate (Cream 0.05%); Fluticasone propionate (Ointment 0.005%); Mometasone furoate (Cream 0.1%); Triamcinolone acetonide (Cream, ointment 0.1%);
V. Lower-Medium Potency: For example:
   Hydrocortisone butyrate (Cream, ointment, solution 0.1%); Hydrocortisone probutate (Cream 0.1%); Hydrocortisone valerate (Cream, ointment 0.2%); Prednicarbate (Cream 0.1%);
VI. Low Potency: For example:
   Alclometasone dipropionate (Cream, ointment 0.05%); Desonide (Cream, gel, foam, ointment 0.05%); Fluocinolone acetonide (Cream, solution 0.01%);
VII. Lowest Potency: For example:
   Dexamethasone (Cream 0.1%); Hydrocortisone (Cream, lotion, ointment, solution 0.25%, 0.5%. 1%); Hydrocortisone acetate (Cream, ointment 0.5%-1%).

The inventors unexpectedly find that the administration of a combination of a kappa opioid receptor agonist and a topical corticosteroid to a patient suffering from atopic dermatitis provides a synergistic therapeutic effect.

### EXAMPLES

### Example 1: Mouse Model of Atopic Dermatitis

In NC/Nga mice, a model of naturally occurring AD, a significant dose-dependent decrease in scratching was observed in Difelikefalin-treated mice over a 90-minute period of observation, compared to the vehicle group.

Similarly, a rapid and significant antipruritic effect of Difelikefalin was observed in the MC903 AD mouse model (Kim B S, et al. Oral Difelikefalin Reduces Pruritus in Atopic Dermatitis [oral presentation]. Presented at the 30th European Academy of Dermatology and Venereology (EADV) Congress; 29 Sep.-2 Oct. 2021). Analyses in this model indicate that expression and activation of the Difelikefalin target receptor is on sensory neurons.

### Example 2: Dose-Ranging Clinical Phase 2 Study to Evaluate Oral Difelikefalin

Oral Difelikefalin 0.25 mg, 0.5 mg, 1.0 mg, or placebo was administered twice daily (BID) in subjects with atopic dermatitis (baseline validated Investigator's Global Assessment scale for AD: vIGA-ADTM≥2 and body surface area [BSA]≤30%) and moderate-to-severe AD-related pruritus (mean baseline Itch Numerical Rating Scale [I-NRS, 0-10 scale] score ≥5.0). The primary and key secondary endpoints were the change from baseline in the weekly mean of the daily I-NRS score at Week 12, and the proportion of subjects achieving at least a 4-point improvement from baseline in I-NRS score at Week 12, respectively.

In this Phase 2 study, numerical improvement in pruritus as measured by the primary endpoint was observed across all 3 doses but the treatment difference from placebo was not statistically significant for the Intent to Treat (ITT) population at Week 12. However, a significant antipruritic effect was observed in subjects with mild-to-moderate AD (BSA<10%). At Week 12, the LS mean treatment group difference in the weekly mean of the daily I-NRS score (Difelikefalin minus placebo) was statistically significant for the combined Difelikefalin group (−0.73; p=0.039). This effect was seen early (Week 1) and sustained through the 12-week Treatment Period.

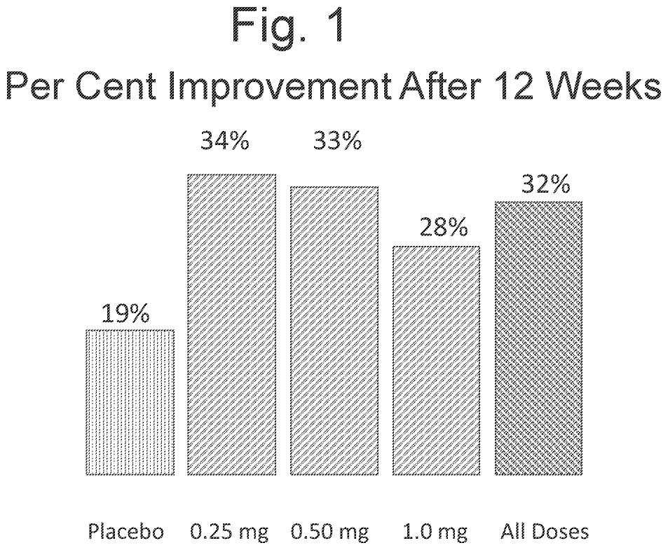

Similarly, a significantly greater proportion of subjects on the combined Difelikefalin group achieved at least a 4-point improvement in I-NRS score at Week 12 compared to placebo (32% vs 19%; p=0.033; non-responder imputation analysis) (See FIG. 1). In addition, a statistically significant difference was observed at Week 12 in the Difelikefalin 0.5 mg group compared to placebo (p=0.046). A trend towards a statistically significant difference was also observed at Week 12 in the Difelikefalin 0.25 mg group (p-value: 0.058).

Estimated percentage and P-value based on a logistic regression model with terms for treatment group and baseline I-NRS score. Subjects who discontinue early or took rescue medication, or have missing data at Week 12, are considered as “non-responders”.

The results observed in subjects with mild-to-moderate AD are supported by numerical improvements in quality of life and sleep with Difelikefalin treatment as measured by the proportion of subjects who achieved a 4-point improvement in DLQI and the proportion of subjects who achieved a 3-point improvement in Sleep NRS, respectively. Modest improvement in skin lesions were observed at Week 12 with Difelikefalin treatment, as measured by the exploratory endpoint of Percent Change in EASI from baseline (0.25 mg −54%; 0.5 mg −44%; 1.0 mg −39%; combined Difelikefalin −46% vs PBO −36%). Pruritus-related and Th2 pathway markers were downregulated in subjects treated with Difelikefalin treatment but not with placebo in the Skin Biopsy sub-study (n=40).

Overall, treatment with oral Difelikefalin was generally well-tolerated. The most common Treatment Emergent Adverse Events (TEAEs) (≥5% in any dose group) were abdominal pain, nausea, dry mouth, headache, dizziness and hypertension. The majority of reported TEAEs were mild or moderate in severity. There were no deaths in the study. Abdominal pain was the most common TEAE leading to study drug discontinuation in the 1.0 mg dose group (6.5%), compared to placebo (1.6%), 0.25 mg (0%) and 0.5 mg (0%) dose groups.

### Example 3: Clinical Trial to Evaluate Oral Difelikefalin as Adjunct Therapy to Topical Corticosteroid for Moderate to Severe Pruritus in Adult Subjects with Atopic Dermatitis

On Day 1, the first dose of oral study drug is administered at the study site after all baseline assessments and PRO measures have been completed using an electronic diary, except for the I-NRS that is completed after oral study drug administration. Subjects are asked to self-administer all subsequent twice-daily doses of the oral study drug with a cup of water at least 2 hours before a meal and around the same time of day. Subjects are requested to record their intake of oral study drug in the electronic diary each day during the Double-blind Treatment Period.

A mid-potent TCS or vehicle cream is provided to be applied by study subjects to skin lesions once a day until control is achieved, then as needed. The amount of TCS or vehicle cream used is monitored throughout the study.

Subjects continue to apply an emollient of their choice (except those containing urea, camphor, and menthol) on their skin at the same frequency (once or twice daily) throughout the study. Rescue medication (defined as a treatment for AD other than emollients or low-to-mid-potent TCS) to control unacceptable signs or symptoms of AD can be used at the discretion and clinical judgment of the Investigator physician. Subjects who take topical rescue medication continue in the study if the topical rescue medication is started at Week 4 or later. However, subjects who require systemic rescue medication discontinue study treatment and discontinue from the study.

Efficacy with respect to pruritus intensity is assessed using I-NRS. Efficacy with respect to impact on sleep, quality of life, skin pain and overall impression of change is assessed using the Patent Reported Outcomes (PROs) for Sleep Disturbance Numerical Rating Scale (NRS), Dermatology Quality of Life Index (DLQI), Skin Pain NRS and Patient Global Impression of Change (PGIC), respectively. The effect on AD skin lesions is assessed using vIGA-ADTM (Validated Investigator's Global Assessment Scale for AD), Eczema Area and Severity Index (EASI) and Body Surface Area (BSA). The effect on work productivity is assessed using the Work Productivity and Impairment Questionnaire: Specific Health Problem (WPAI:SHP).

The Itch Numerical Rating Scale ((I-NRS), Sleep Disturbance NRS, Skin Pain NRS, Dermatology Quality of Life Index (DLQI), Work Productivity and Impairment Questionnaire: Specific Health Problem (WPAI:SHP) and Patient Global Impression of Change (PGIC) questionnaires are completed by the subject, and the vIGA-ADTM, EASI and BSA assessments are performed by the Investigator physician.

The specifications of each of the U.S. patents and published patent applications, and the texts of the literature references cited in this specification are herein incorporated by reference in their entireties. In the event that any definition or description found in one or more of these references is in conflict with the corresponding definition or description herein, then the definition or description disclosed herein is intended.

The examples provided herein are for illustration purposes only and are not to be interpreted as limiting the scope of the invention, the full scope of which will be immediately recognized by those of skill in the art.

We claim:

1. A method of treatment of atopic dermatitis in a patient suffering from atopic dermatitis, the method includes administering a course of treatment comprising: administering an effective amount of a kappa opioid receptor agonist (KORA) orally to the patient and applying an effective amount of a topical corticosteroid (TCS) to an area of the patient affected by the atopic dermatitis.

2. The method of claim 1, wherein the KORA is administered orally to the patient and the TCS is applied to an area of the patient affected by the atopic dermatitis on the same day.

3. The method of claim 2, wherein the KORA is administered orally one or more times per day to the patient and the TCS is applied to an area of the patient affected by the atopic dermatitis once per day.

4. The method of claim 2, wherein the KORA is administered orally twice per day to the patient.

5. The method of claim 4, wherein the course of treatment is for 1-7 days.

6. The method of claim 4, wherein the course of treatment is for 8-84 days.

7. The method of claim 4, wherein the course of treatment is for 84 days or more.

8. The method of claim 3, wherein the course of treatment comprises applying the TCS to the area of the patient affected by the atopic dermatitis daily until control of the atopic dermatitis of the affected area is achieved and subsequently as needed for 7-84 days.

9. The method of claim 1, wherein the course of treatment comprises applying the TCS to the area of the patient affected by the atopic dermatitis daily until control of the atopic dermatitis of the affected area is achieved and subsequently as needed for 84 days or more.

10. The method of claim 1, wherein the KORA is administered in an oral formulation selected from an oral formulation selected from a powder, a tablet, a capsule, a gelcap, an amorphous solid, a crystalline form, a liquid, a slurry and a gel.

11. The method of claim 10, wherein the oral formulation comprises from 0.01-10.0 mg Difelikefalin.

12. The method of claim 11, wherein the oral formulation comprises from 0.1-5.0 mg Difelikefalin.

13. The method of claim 12, wherein the oral formulation comprises from 0.2-1.0 mg Difelikefalin.

14. The method of claim 13, wherein the oral formulation comprises from 0.25-0.5 mg Difelikefalin.

15. The method of claim 1, wherein the TCS is selected from the group consisting of a very potent corticosteroid, a potent corticosteroid, a medium potency corticosteroid, a low potency corticosteroid or a lowest potency corticosteroid in the form of an ointment, a cream, a gel, a lotion, a foam or a solution, and having a concentration of from 0.005% to 1% (w/w).

16. The method of claim 15, wherein the very potent corticosteroid is selected from the group consisting of augmented betamethasone dipropionate, clobetasol propionate; diflorasone diacetate and halobetasol propionate.

17. The method of claim 15, wherein the potent corticosteroid is selected from the group consisting of amcinonide, augmented betamethasone dipropionate, betamethasone dipropionate, desoximetasone, desoximetasone, diflurasone diacetate, flurocinonide, halcinonide, mometasone furoate and triamcinolone acetonide.

18. The method of claim 15, wherein the medium potency corticosteroid is selected from the group consisting of betamethasone valerate, clocortolone pivalate, desoximetasone, fluocinolone acetonide, flurandrenolide, fluticasone propionate, mometasone furoate and triamcinolone acetonide.

19. The method of claim 15, wherein the lower-medium potency corticosteroid is selected from the group consisting of hydrocortisone butyrate, hydrocortisone probutate, hydrocortisone valerate and prednicarbate.

20. The method of claim 15, wherein the low potency or lowest potency corticosteroid is selected from the group consisting of alclometasone dipropionate, desonide, fluocinolone acetonide, dexamethasone, hydrocortisone and hydrocortisone acetate.
